(19) United States
(12) Patent Application Publication
(10) Pub. No.: US 2024/0252703 A1
(43) Pub. Date: Aug. 1, 2024

(54) MODULAR MOBILE SERVICE ROBOT AND ACCESSORIES FOR EFFICIENT SITE DISINFECTION VIA GERMICIDAL UVC LIGHT

(71) Applicant: Loop Robots B.V., Rijswijk (NL)

(72) Inventors: Per Slycke, Rijswijk (NL); Maarten SLUYTER, Rijswijk (NL); Kim NICOLAI, Rijswijk (NL); Gustaaf Liqui LUNG, Rijswijk (NL); Dimitrios CHRONOPOULOS, Rijswijk (NL); Aswin CHANDARR, Rijswijk (NL)

(21) Appl. No.: 17/229,793

(22) Filed: Apr. 13, 2021

(51) Int. Cl. A61L 2/24; A61L 2/10

(52) U.S. Cl. CPC A61L 2/24; A61L 2/10; A61L 2202/11; A61L 2202/14; A61L 2202/16; A61L 2202/25

(57) ABSTRACT

A site disinfection robot includes a robot base and one or more UVC light sources with reflectors. The one or more UVC light sources may be included in a tower that is detachable from the base. The tower may asymmetrically locate the one or more UVC light sources along one side of the robot to allow efficient, close disinfection of surfaces. Additional UVC light sources may be provided for disinfection of surfaces under overhangs and of the robot itself including its wheels. A portable accessory, dockable to the robot base, may be provided to allow additional manual disinfection by a human operator.

### TECHNICAL FIELD

The present disclosure is related generally to mobile service robots and, more particularly, to a mobile service robot for site disinfection.

### BACKGROUND

There are many environments wherein frequent and effective disinfection of indoor spaces is required. For example, in the hospital environment, preventing Hospital Acquired Infections (HAI) is an ongoing challenge that is critical to patient safety. In addition to the patient cost, the associated financial costs are enormous. In acute care hospitals in the U.S. alone, such costs are approximately 97-147 Billion USD annually. One in ten patients worldwide is infected by an antibiotic-resistance pathogen while being treated in a hospital. This statistic does not include infections by viruses, such as SARS, MERS, SARS-COV-2 or influenza. HAI lead to 3 million infections in the USA alone, causing 48.000 preventable deaths each year.

Ultra-violet C (UVC) light from low-pressure mercury lamps, emitting strong radiation at 253.7 nm, is a well-known technology for inactivating pathogens. The germicidal efficacy of UVC is due to the fact that UVC directly disrupts the DNA or RNA in the pathogen. When enough damage is caused to the RNA or DNA, the pathogen can no longer multiply and is thus inactivated. UVC has many advantages over traditional methods for surface and room scale disinfection. It does not use chemicals and therefore does not cause resistance in pathogens or harm to the environment or personnel carrying out the disinfection process. Moreover, unlike some manual touch disinfectants like chlorine and non-touch disinfectants like hydrogen-peroxide vapor that can damage materials and surfaces, UVC like does not leave any residues and does not damage materials at low dosages.

Typical required UVC doses to achieve a desired reduction in active pathogens are shown in the following table in mJ/cm2. This table is from Malayeri, Adel & Mohseni, Madjid & Cairns, Bill. (2016). Fluence (UV Dose) Required to Achieve Incremental Log Inactivation of Bacteria, Protozoa, Viruses and Algae. IUVA News. 18. 4-6.

| Name | Log1 | Log2 | Log3 | Log4 |
|---|---|---|---|---|
| S Aureus (MRSA) | 4.50 | 7.20 | 8.80 | 10.00 |
| S Aureus (MSSA) | 4.40 | 5.80 | 6.40 | 7.30 |
| Acinetobacter Baumanii | 1.80 | 3.60 | 6.20 | 9.00 |
| Pseudomonas aeruginosa (MDR) | 1.50 | 2.60 | 3.80 | 5.00 |
| Enterococcus faecium | 7.00 | 9.00 | 11.00 | 13.00 |
| Enterococcus faecalis | 3.70 | 8.00 | 14.00 | 18.00 |
| Norovirus | 10.00 | 15.00 | 22.00 | 27.00 |
| Klebsiella terrigena | 3.60 | 6.40 | 9.30 | 12.00 |
| Sars Cov2 | 3.00 | 5.00 | 10.00 | 15.00 |
| Clostridium difficile | 16.00 | 34.00 | 46.00 | 62.00 |

Despite the many advantages of UVC, it is not yet widely applied because of a few key challenges. UVC light is only germicidal on surfaces that are irradiated by the UVC light. Any surfaces in shadow from the UVC light will not be disinfected. In addition, UVC is only germicidal if an adequate dosage is delivered to each surface that needs to be disinfected. The required dosage is different for each pathogen, since each pathogen exhibits a unique level of sensitivity to damage from UVC light. For this reason, UVC lamps used to disinfect surfaces and rooms in a healthcare setting have been designed to be movable in an attempt to reduce shadows and get closer to objects that need to be disinfected.

However, UVC lamps consume very significant amounts of electrical energy. A typical moveable UVC system is powered from an electrical outlet and consumes about 1-2 kW of electrical energy and needs to be powered for a significant amount of time (e.g., 30-60 min) to try to reach sufficient UVC dose delivered to space and surfaces furthest away from the lamps. For each disinfection, an operator can typically move the UVC lamps around a few times in a room to try to eliminate any shadows caused by objects in a room, such as a bed, chair or equipment. This is a laborious and error prone process. Recently, leveraging mobile service robot technology, several mobile robots have been developed that can automate the process of moving the UVC lamps around in the room to several locations, to minimize the amount of shadows and speed up operations somewhat. These systems however are limited in their use because the operation time is very limited (typically around 2 hours) and the robot is required to carry very large batteries. This results in robot UVC systems being expensive, heavy and slow to charge before being able to carry out a subsequent disinfection cycle.

Germicidal UVC lamps can be manufactured via different methods and incorporating different technologies. For example, germicidal lamps may be high pressure mercury lamps, amalgam lamps with specific properties or may use different mechanisms all together to generate (UV) light. For example, LEDs can be used to generate radiation at wave lengths other than 253.7 nm (which is specific for low pressure mercury lamps). In addition, far UVC lamps operating at 222 nm have also been shown to have germicidal effects. In this disclosure, UVC lamps should be construed to include all of these types of lamps and any other lamp technology that has a germicidal effect.

Before proceeding to the remainder of this disclosure, it should be appreciated that the disclosure may address some of the shortcomings listed or implicit in this Background section. However, any such benefit is not a limitation on the scope of the disclosed principles, or of the attached claims, except to the extent expressly noted in the claims.

Additionally, the discussion of technology in this Background section is reflective of the inventors' own observations, considerations, and thoughts, and is in no way intended to be, to accurately catalog, or to comprehensively summarize any prior art reference or practice. As such, the inventors expressly disclaim this section as admitted or assumed prior art. Moreover, the identification or implication herein of one or more desirable but unfollowed courses of action reflects the inventors' own observations and ideas, and should not be assumed to indicate an art-recognized desirability.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a schematic drawing of an example disinfection robot within which embodiments of the disclosed principles may be implemented;

FIG. 2 is a side-view of an example light configuration on the robot of FIG. 1 in accordance with an embodiment of the disclosed principles;

FIG. 3 is a front view of another example light configuration on the robot of FIG. 1 in accordance with an embodiment of the disclosed principles;

FIG. 4 is a radiation/distance plot showing an example effect on dosage delivered as a percentage of dosage delivered from placement of tower in the middle of the robot, for example 30 cm from the edge, depending upon tower placement in accordance with an embodiment of the disclosed principles;

FIG. 5 is a radiation/distance plot showing the effect on dosage delivered at a surface on varying distances from the robot after a certain amount of time, for example 10 seconds, depending upon tower placement and robot navigation in accordance with an embodiment of the disclosed principles;

FIG. 6 is a schematic view of a modular design in accordance with an embodiment of the disclosed principles, wherein the UVC tower can be removed from the base;

FIG. 7A is a schematic top view of a low dosage wide area coverage tower head, with a lamp on each of 4 corners in accordance with an embodiment of the disclosed principles;

FIG. 7B is a schematic top view of a high dosage small area coverage tower head with three lamps on one side in accordance with an embodiment of the disclosed principles;

FIG. 8A is a schematic top view of a configuration wherein one or more UVC lights are mounted in the bottom-sides of the robot in accordance with an embodiment of the disclosed principles;

FIG. 8B is a schematic frontal view of a configuration wherein one or more UVC lights are mounted in the bottom-sides of the robot in accordance with an embodiment of the disclosed principles;

FIG. 9 is a schematic frontal view of a configuration wherein one or more UVC lights are mounted near a wheel of the robot in accordance with an embodiment of the disclosed principles;

FIG. 10 is a schematic side view of a configuration wherein one or more UVC lights are mounted at multiple heights on the robot in accordance with an embodiment of the disclosed principles;

FIG. 11 is a schematic side view of a configuration wherein one or more mirrors having high UVC reflectivity is located at the robot docking station to disinfect the exterior of the robot while charging in accordance with an embodiment of the disclosed principles;

FIG. 12 is a schematic side view of a configuration wherein one or more mirrors having high UVC reflectivity are permanently placed in a site to enable UVC light reflect into otherwise unreachable locations in accordance with an embodiment of the disclosed principles;

FIG. 13 is a cross-sectional side view of a portable accessory usable in conjunction with a disinfection robot in accordance with an embodiment of the disclosed principles; and

FIG. 14 is a cross-sectional side view of a portable accessory usable in conjunction with a disinfection robot in accordance with an embodiment of the disclosed principles.

### DETAILED DESCRIPTION

Before presenting a detailed discussion of embodiments of the disclosed principles, an overview of certain embodiments is given to aid the reader in understanding the later discussion. As noted above, many situations and environments require energy-efficient and effective disinfection of indoor spaces. Typical sites include hospital and other healthcare environments, although other environments such as convalescent and food preparation environments also require high hygienic standards and minimum microbial contamination to avoid adverse human health effects.

While UVC light from low pressure mercury lamps emitting at 253.7 nm is known for inactivating pathogens at appropriate dosage levels, UVC disinfection can be time-consuming and energy-inefficient due to slow charging of mobile light sources, wasted radiation, high electrical energy consumption, and consequent limited operation time.

In an embodiment of the disclosed principles, an asymmetric robot design in employed, with emission from the robot being selective rather than uniform. For example, the robot contains a tower where UVC light sources with reflectors (e.g., either LP mercury lamps or UVC LEDs) may be placed on one side of the robot. This design focuses all available UVC power to one side of the robot (the active side), enabling the robot to approach surfaces to be disinfected along this active side. This in turn permits the robot to deliver the required UVC power on surfaces without wasting energy on other sides.

In a further embodiment of the disclosed principles, the UVC light sources may be placed as close as possible to the outermost side edges of the robot. In a further embodiment of the disclosed principles, ranging sensors (i.e., 3D cameras/3D LIDARs) are placed on the top of the tower, facing downward on the sides. This configuration provides a complete view of 270 degrees (front, side with lamps and rear) in the vicinity of the robot. With a detailed 3D view of its immediate surroundings, the robot can navigate with its lamps very close (5-10 cm) to the surfaces to be disinfected.

In another embodiment, a modular design is provided, wherein the UVC tower can be removed from the base for easy transport. With this feature, the robot and tower can be transport without needing special high clearance vehicles. The tower may be an independent unit comprising light sources and compatible electronic controllers. This tower is electrically coupled with the robot base through heavy duty power connectors available off-the-shelf, providing power to drive the lamps and signals to control the lamps. Female connectors are present at the robot base to prevent accidental contact with human users, when the tower is not connected. The electrical mating connectors are supported and held in place through mechanical support locks that can be easily removed. The connectors may include an electrical jumper on both male and female sides which provide electrical continuity only when the tower is attached to the base. This signal is connected to relays on the base. Such configuration provides implicit safety by enabling power to pass through the joint only when a tower is connected.

In a further aspect, a modular design wherein different configurations of the UVC tower are provided based on application type, i.e., low dosage disinfection on large areas as opposed to high dosage disinfection in small areas. Different tower heads can thus be mounted on a same base.

In yet a further aspect, UVC lights are placed in the bottom and/or sides of the robot, facing upwards to reach surfaces such as the bottom side of beds, tables, trolleys etc. Such placement of lamps on a robot allows for disinfection of these not-commonly used areas as well, when needed. Moreover, UVC lamps or LEDs under the robot or integrated in the wheel hubs can disinfect the wheels to prevent the wheels from carrying pathogens from one space to another. In an embodiment, lamp sources are placed at multiple heights to provide sufficient dosage at heights ranging from a significant height in human dwellings, where objects and surfaces may be present that needs to be disinfected such as 2 m, down to the ground level.

A mirror or multiple mirrors (having high UVC reflectivity) at the docking station may be used to disinfect the exterior of the robot while charging, if such areas are not disinfected by the lamps themselves while in regular use. Optionally this could be configured in a separate cabinet. Permanent placement of UVC reflective mirrors, materials or paint in certain places in a room may be used to enable UVC light to reach places that are physically not possible to reach directly from the robot.

In a further aspect of the disclosed principles, a hand-held self-contained disinfection unit is provided containing one or more UVC light sources. A battery and control circuitry are provided. A visual sensing system (wide angle cameras/3D camera/LIDAR), an inertial measurement unit and a wireless radio communication system (WiFi/Bluetooth/etc.) may also be provided. The hand-held self-contained disinfection unit can be mounted and charged from the host robot, and may communicate with the host robot using a wired or wireless radio link. This accessory may have a reflector such that the UVC light is directed away from the user. A human operator can use the hand-held self-contained disinfection unit to manually shine UVC light on required surfaces and receive guidance by audio or visual means on where to apply disinfection (which spatial location) and for how long.

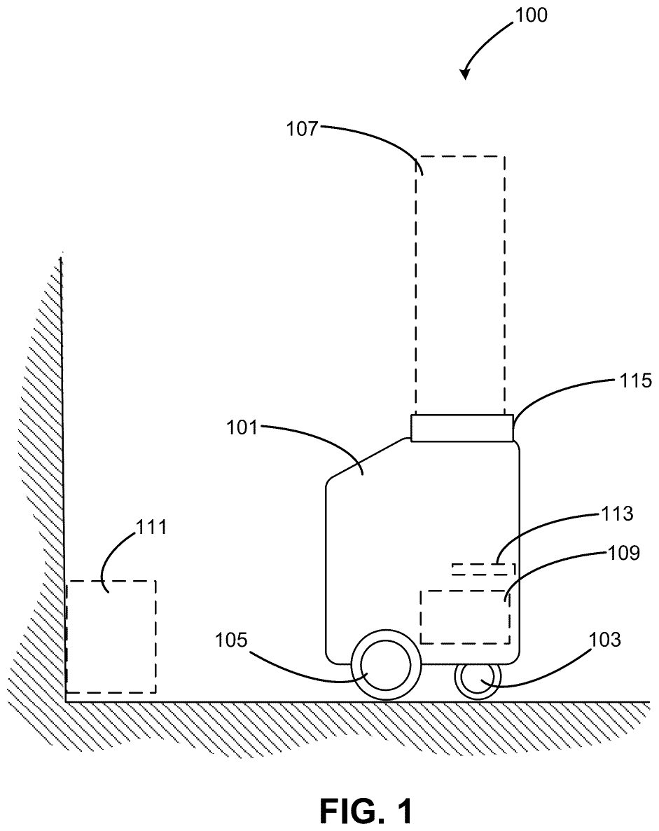

With this general overview in mind, and turning now to a more detailed discussion in conjunction with the attached figures, the techniques of the present disclosure are illustrated as being implemented in or via a suitable device environment. Thus, for example, FIG. 1 illustrates an example disinfection robot which may embody one or more aspects of the disclosed principles.

In the illustrated embodiment, the disinfection robot 100 includes a base 101, which is supported, driven and steered by wheels 103, 105. The disinfection robot 100 includes a light tower 107, which holds one or more UVC emitting lights. The illustrated tower 107 is merely to indicate a region wherein lights may be mounted and is not intended to illustrate a particular shape or number of lights. It will be appreciated that the disinfection robot 100 may include UVC emitting lights in other positions in addition to or instead of the light tower 107.

The disinfection robot 100 includes a power source 109 such as a battery, for powering the navigation of the disinfection robot 100 as well as the one or more UVC emitting lights. A wall charging station 111 may be provided for periodically recharging the power source 109. The disinfection robot 100 further includes a processor system 113 for executing the robot-based activities discussed herein. A collar 115 may be used to enable physical and electrical separation of the light tower 107 from the main body of the robot 101. Necessary or desirable peripheral systems such as sensors, LIDAR sensors, cameras, motion tracking sensors, computer memory, latches, switches, antennas, communications facilities and so on are also included in the disinfection robot 100 but are omitted from the figure for clarity.

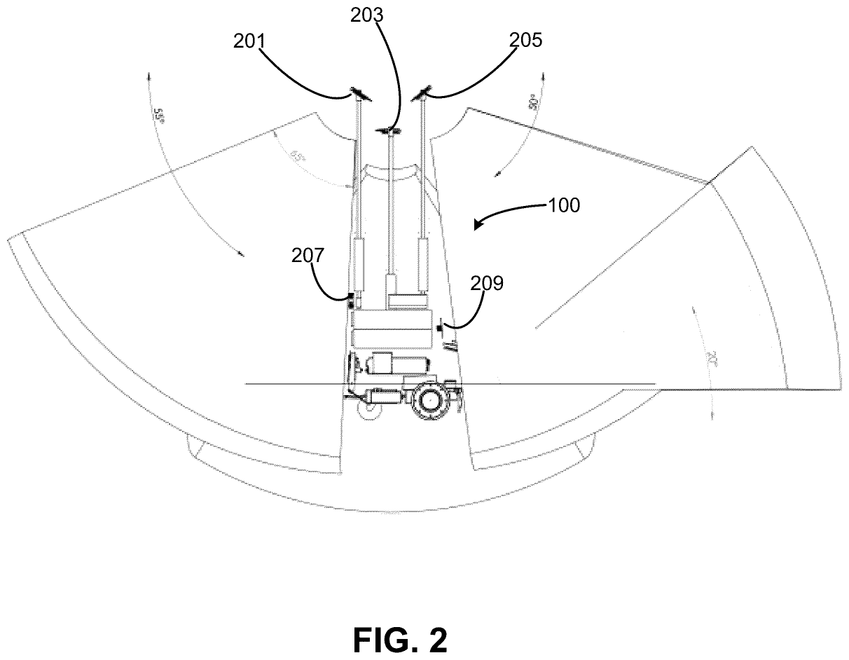

Turning to FIG. 2, this figure is a side-view of an example light configuration on the robot, e.g., the robot of FIG. 1. In the illustrated configuration, three lamps 201, 203, 205 are placed at different heights to enable delivery of UVC light dosage at a wide height range, from the floor to about 180 centimeters or more. The illustrated embodiment also includes 3D cameras placed facing forward 207, rearward 209, and sideward (not shown) providing coverage in 270 degrees around the robot.

This is an example of asymmetric robot design, with lights placed asymmetrically around the robot's central axis. As in FIG. 1, the UVC light sources and reflectors are contained a tower on one-side of the robot. This design focusses all the UVC power available to the robot in one side (the active side) enabling the robot to approach surfaces along this active side to deliver the required UVC power without wasting energy on other sides of the robot.

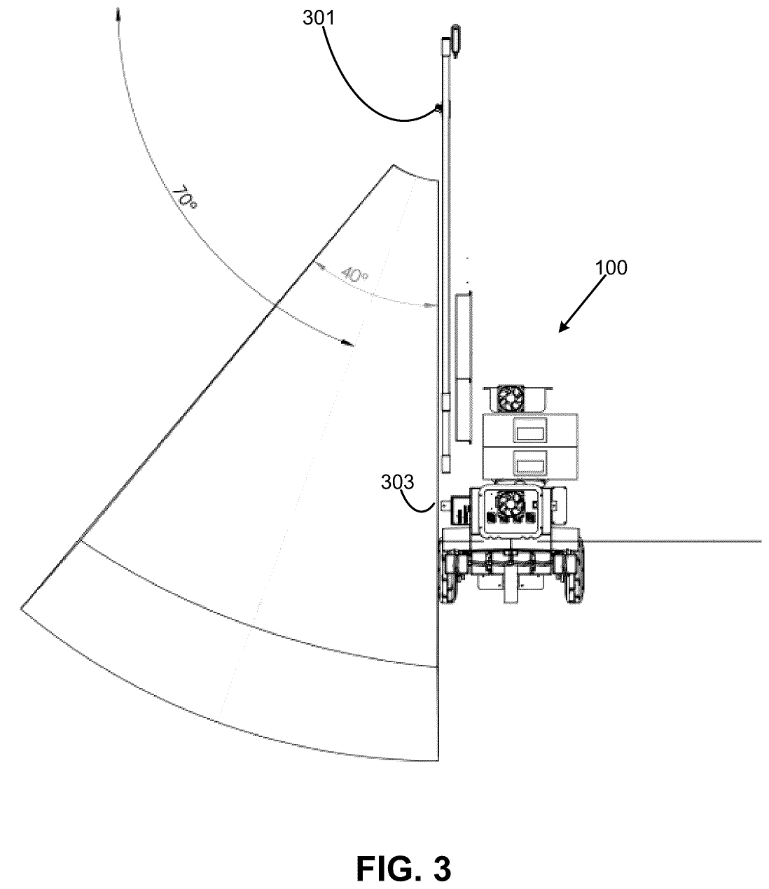

FIG. 3 shows another example of asymmetric lamp placement, with the lamp(s) 301 being placed on a side edge of the robot 100. It also shows an example 3D camera 303 on the side providing detailed 3D view of immediate surrounding of the robot. In this way, the UVC light sources are placed as close as possible to the outermost side edges of the robot.

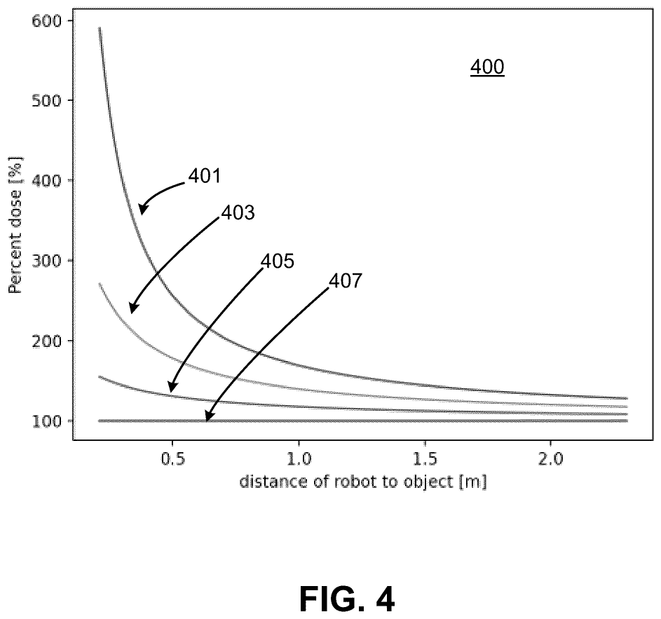

FIG. 4 shows a relative radiation/distance plot 400 for an example configuration of UVC lamps and objects at various distances from a light source on the robot. The first trace 401 shows a plot of the percentage of UVC dosage delivered with the tower 0 cm from robot edge compared to the tower being placed close to the center of the robot at for example 30 cm from the edge of the robot, the second trace 403 shows a plot of the percentage with the tower 10 cm from robot edge, the third trace 405 shows a plot of the percentage with the tower 20 cm from robot edge, and the fourth trace 407 shows a plot of the reference position with the tower 30 cm from robot edge (thus by definition 100%). As can be seen, for example, an object at a distance of 0.5 m from the robot with the UVC light source 0 cm from the edge creates almost 300% increase in efficiency as compared to placing the light source 30 cm inside from the edge of the robot. It should also be noted that the relatively small difference in UVC lamp location (tower) on the robot also has a significant effect in the efficiency at longer distances to objects, such as a 30-40% increased efficiency at 2 m distance to object when the UVC lamps are placed at the edge relative to being 30 cm from the edge of the robot, which is only a 15% difference.

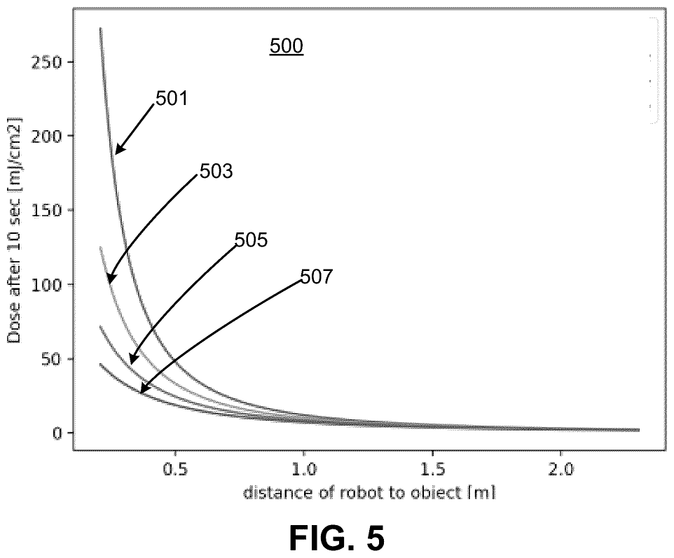

In a further aspect, ranging sensors (3D cameras/3D LIDARs) are located on the top of the tower, facing downwards on the sides. This configuration of sensors and the robot provides a complete view of 270 Degree (front, side with lamps and rear) in the vicinity of the robot. With such a detailed 3D view of its immediate surroundings, the robot can navigate with its lamps very close (e.g., 5-10 cm) from the surfaces to be disinfected. The combined effect of optimum lamp placement and optimum navigation can be seen in the plot 500 of FIG. 5, which shows dosage delivered over 10 seconds, with robot at 20 cm from the surface of interest, at varying lamp inset distances. In particular, trace 501 shows a plot with the tower 0 cm from robot edge, trace 503 shows a plot with the tower 10 cm from robot edge, trace 505 shows a plot with the tower 20 cm from robot edge, and trace 507 shows a plot with the tower 30 cm from robot edge.

As can be seen, when the tower is placed at the edge of the robot (0 cm from robot edge, trace 501), there is a 5 fold, 500% increase (from 50 mJ/cm² to 250 mJ/cm²) in UVC energy delivery, when the robot navigates at 20 cm from the surface compared to 30 cm to the surface.

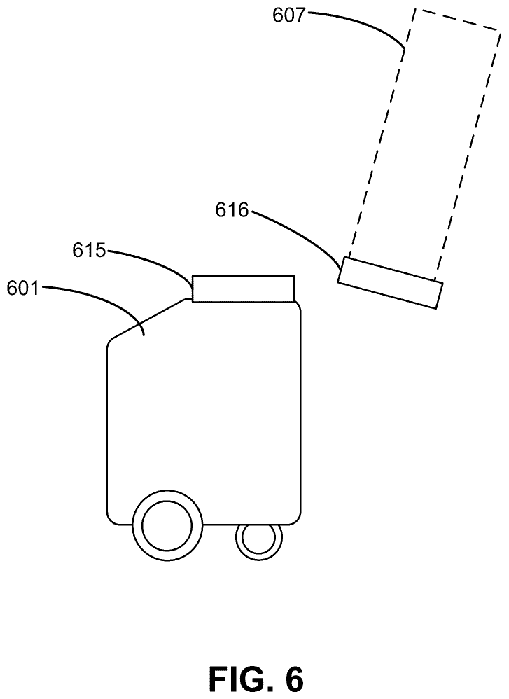

It was noted above that a modular design is provided in an embodiment, wherein the UVC tower can be removed from the base for easy transport. With this modularity, the robot and tower can, for example, be transported without needing special high clearance vehicles. An example of this feature is shown in FIG. 6. As can be seen, the tower 607 (see also 107 of FIG. 1) is an independent unit comprising light sources and compatible electronic controllers.

In this embodiment, the tower 607 is electrically couplable with the robot base 601 (see also 101 of FIG. 1) through off-the-shelf heavy-duty power connectors within connection collars 615, 616, providing AC power to drive the lamps and signals to control the lamps through, e.g., via an Ethernet connection or otherwise. In an embodiment, female rather than male connectors are used at the base connection collar 615 to prevent accidental contact with human users, when the tower is disconnected. The electrical mating connectors are supported and held in place through mechanical support locks that can be easily removed.

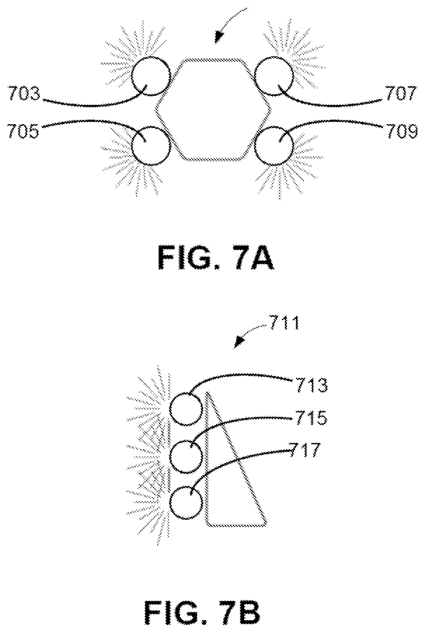

This modular design allows different configurations of the UVC tower to be placed on the base depending upon the intended application and site complexities. FIG. 7A shows a top view of a low dosage wide area coverage tower head 701, with a lamp (703, 705, 707, 709), e.g., of 130 W, on each of 4 corners. In contrast, FIG. 7B shows a top view of a high dosage small area coverage tower head 711 with three lamps (713, 715, 717), e.g., of 200 W, on one side.

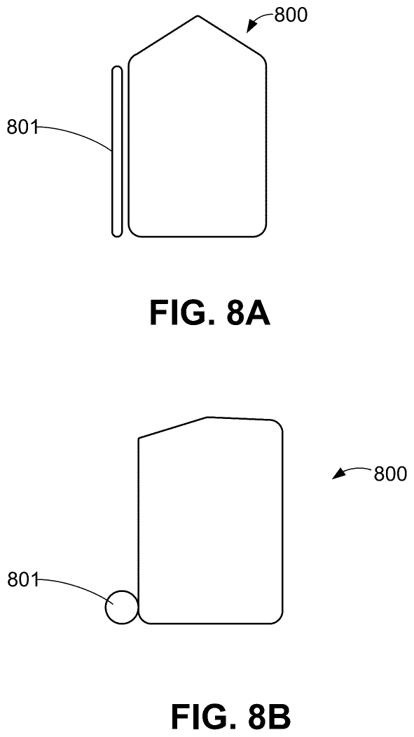

FIGS. 8A and 8B show a configuration wherein one or more UVC lights 801 are mounted in the bottom-sides of the robot 800, facing upwards to reach surfaces such as bottom side of beds, tables, trolleys etc. Such placement of lamps on a robot allows for disinfection of these not-commonly used areas as well, when needed. FIG. 8A is a top view of such configuration, while FIG. 8B is a frontal view of such configuration.

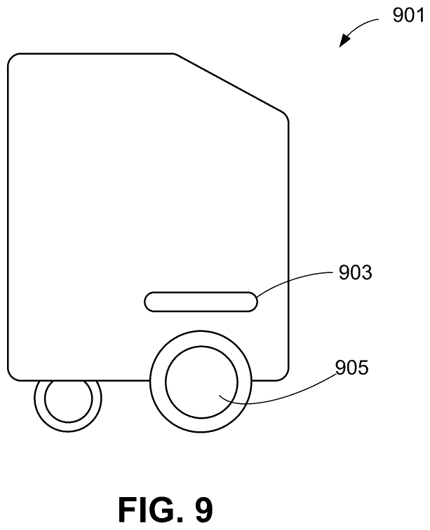

FIG. 9 show the use of UVC lamps/LEDs 903 near the wheel 905 of the robot 901 to disinfect the wheels and prevent the wheels from carrying pathogens from one space to another. It will be appreciated that a corresponding lamp may also be placed on the opposite side of the machine (not shown). Lamps can also or alternatively be placed under the robot or integrated in the wheel hubs.

Disinfection robots generally operate in areas with high infection risk. While the robot performs its tasks of disinfecting the surfaces in its environment, the bio-contaminants may stick to the wheels of the robot and be transported to other areas. The foregoing placement of additional lamps places a low power UVC light source at a close proximity to the wheels, fenders and bottom surface of the robot, to constantly disinfect the area and prevent cross contamination between sites.

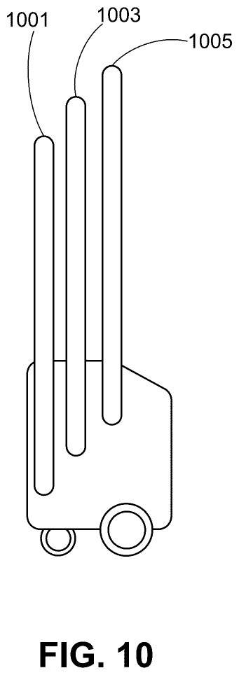

Most surfaces that need to be disinfected are in the hand height level for humans. However, there are places, especially in hospital environments, and operating rooms where objects such as overhead lamps, etc. can hang from the ceiling or otherwise be placed out of range. In these cases, referring to FIG. 10, placement of lamp sources 1001, 1003, 1005 at multiple heights can provide sufficient dosage at heights ranging from, for example, 1.8 m to the ground.

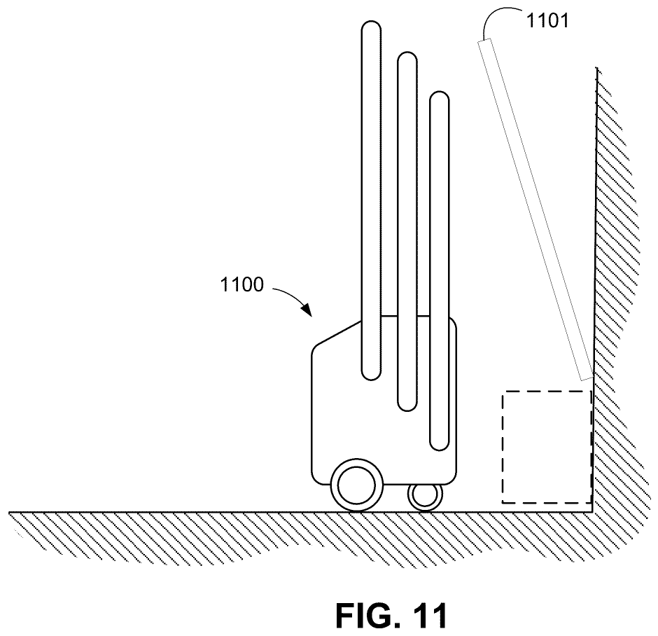

Similar to the self-disinfecting embodiment of FIG. 9, the example of FIG. 11 shows that a mirror 1101 or multiple mirrors (having high UVC reflectivity) can be used at the docking station to disinfect the exterior of the robot 1100 while charging. Optionally this mirror could be configured in a separate cabinet.

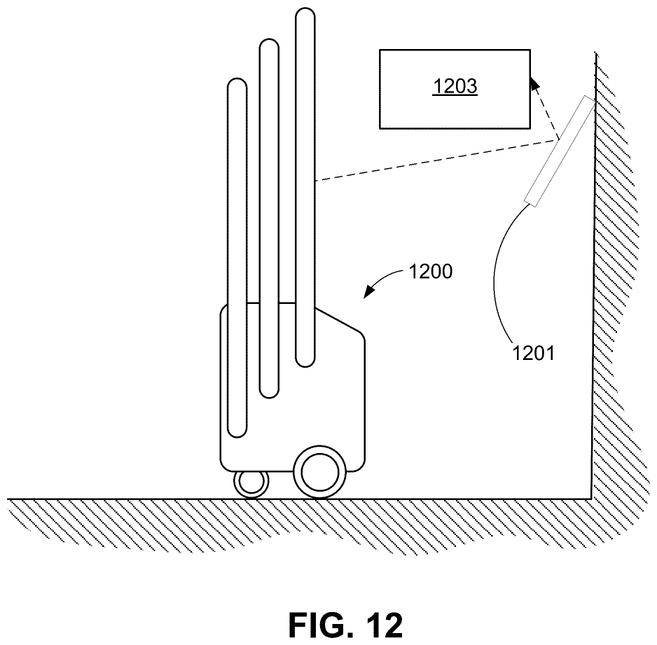

Moreover, in accordance with a further embodiment, permanent placement of UVC reflective mirrors in certain places in a room may enable UVC light to reach places through reflection that are not reachable by direct line-of-sight UVC illumination from the robot. For example, the space behind cabinets, under tables or beds, on top of tall fixtures and so on may be out of line-of-sight range for a robot. FIG. 12 illustrates one such scenario, wherein a permanent UVC reflective mirror 1201 reflects radiation from the lamps of the robot 1200 to disinfect a back surface of a cabinet 1203 that is unreachable by direct line-of-sight illumination.

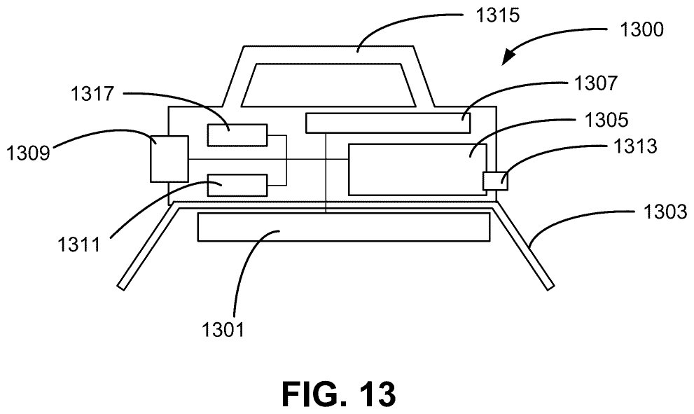

FIG. 13 is a cross-sectional side view of a hand-held self-contained disinfection unit 1300 containing one or more UVC light sources 1301. The one or more UVC light sources are shielded by a shield 1303 to protect the user from prolonged exposure to the light. A battery 1305 and control circuitry 1307 are provided to power and manage the device 1300. A visual sensing system 1309 (e.g., one or more wide angle cameras/3D camera/LIDAR), an inertial measurement unit 1311 and a wireless radio communication system (WiFi/Bluetooth/etc.) may also be provided.

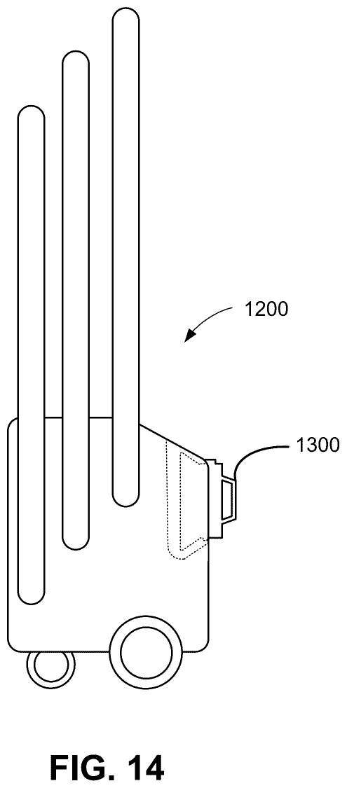

The hand-held self-contained disinfection unit 1300 can be mounted on the host robot 1200 as shown in FIG. 14, e.g., in a cavity or opening, or alternatively on the robot surface, via appropriate latching and electrical connection. The battery 1305 may be charged by the robot, e.g., via a charging port 1313, and may communicate with the host robot using a wired or wireless radio link.

A human operator holding the handle 1315 may use the hand-held self-contained disinfection unit 1300 to manually shine UVC light on required surfaces, and may receive guidance by audio (via speaker 1317) or visual means as to where to apply disinfection (which spatial location) and for how long. With respect to visual guidance, the hand-held self-contained disinfection unit 1300 may include a visual indicator such as a screen or panel. Alternatively or additionally, a virtual reality (VR) or augmented reality (AR) headset may provide direct spatial visual guidance based on 3D spatial data from a prior executed automatic disinfection run by the robot.

It will be appreciated that various systems and processes have been disclosed herein. However, in view of the many possible embodiments to which the principles of the present disclosure may be applied, it should be recognized that the embodiments described herein with respect to the drawing figures are meant to be illustrative only and should not be taken as limiting the scope of the claims. Therefore, the techniques as described herein contemplate all such embodiments as may come within the scope of the following claims and equivalents thereof.

We claim:

1. A robot for site disinfection, the site having surfaces to be disinfected, the robot comprising:
a robot base having a direction of travel, and a left side and a right side with respect to the direction of travel;
one or more UVC light sources with reflectors located substantially at an active side of the robot base, the active side being one of the left side and the right side, the other of the left side and right side being an inactive side; and
a processor configured to navigate the robot to approach surfaces to be disinfected along the active side of the robot, enabling the robot to deliver a required UVC power for disinfection from the one or more UVC light sources, on the surfaces to be disinfected, without wasted energy on the inactive side.

2. The robot for site disinfection in accordance with claim 1, wherein the one or more UVC light sources with reflectors are in a removable tower unit.

3. The robot for site disinfection in accordance with claim 2, wherein the removable tower unit further comprises electronic controllers for the one or more UVC light sources.

4. The robot for site disinfection in accordance with claim 2, wherein the tower unit is electrically couplable with the robot base via one or more power connectors providing power to drive and control the one or more UVC light sources.

5. The robot for site disinfection in accordance with claim 4, wherein the one or more power connectors each include a female connectors at the robot base and a male connector at the tower unit to prevent accidental electrical contact with human users when the tower unit is not connected to the robot base.

6. The robot for site disinfection in accordance with claim 5, wherein the one or more power connectors each include at least one electrical jumper which provides electrical continuity only when the tower unit is attached to the robot base.

7. The robot for site disinfection in accordance with claim 1, wherein one or more additional UVC light sources are placed on the robot so that the one or more additional UVC light sources direct UVC light upward to reach overhanging surfaces.

8. The robot for site disinfection in accordance with claim 1, wherein one or more additional UVC light sources are placed on the robot so that the one or more additional UVC light sources direct UVC light toward one or more wheels of the robot to prevent the wheels from carrying pathogens from one space to another.

9. The robot for site disinfection in accordance with claim 1, wherein the one or more UVC light sources are located at multiple different heights to provide sufficient dosage at a wider range of heights.

10. The robot for site disinfection in accordance with claim 1, further comprising a docking station and at least one UVC-reflective mirror at the docking station to disinfect the exterior of the robot via the one or more UVC light sources during charging.

11. The robot for site disinfection in accordance with claim 1, further comprising at least one permanent UVC-reflective surface in the site to redirect UVC light emitted by the one or more UVC light sources.

12. The robot for site disinfection in accordance with claim 1, further comprising at least one ranging sensor on each of a plurality of sides of the robot facing downward to provide information regarding objects and surfaces in the vicinity of the robot.

13. The robot for site disinfection in accordance with claim 12, wherein the at least one ranging sensor is one of a 3D (three-dimensional) camera and a 3D LIDAR (laser imaging, detection, and ranging) system.

14. An autonomous robot for site disinfection, the autonomous robot comprising:
a robot base having a plurality of wheels and a top surface;
a first electrical connector on the top surface; and
a UVC tower comprising one or more UVC lamps and having a bottom surface, the UVC tower having a second electrical connector on the bottom surface, the second electrical connector being a couplable to the first electrical connector, such that the UVC tower can be installed on the robot base by coupling the first and second connectors and removed from the robot base by uncoupling the first and second connectors.

15. The autonomous robot in accordance with claim 14, wherein the first electrical connector is a female connector and the second electrical connector is a male connector to prevent accidental electrical contact with human users when the tower unit is not connected to the robot base.

16. The autonomous robot in accordance with claim 15, wherein the first and second electrical connectors each include at least one electrical jumper which provides electrical continuity only when the tower unit is attached to the robot base.

17. The autonomous robot in accordance with claim 14, wherein one or more additional UVC light sources are placed on the robot so that the one or more additional UVC light sources direct UVC light toward one or more wheels of the robot to prevent the wheels from carrying pathogens from one space to another.

18. The autonomous robot in accordance with claim 14, wherein the one or more UVC lights are located at multiple different heights to provide sufficient dosage at a wider range of heights.

19. The autonomous robot in accordance with claim 14, further comprising a docking station and at least one UVC-reflective mirror at the docking station to disinfect the exterior of the robot via the one or more UVC light sources during charging.

20. The robot for site disinfection in accordance with claim 14, further comprising at least one permanent UVC-reflective surface in the site to redirect UVC light emitted by the one or more UVC lights.

21. A robot for site disinfection, the robot comprising:
a robot base having wheels, an electrical power source to drive the wheels, and a processor configured to navigate the robot base autonomously;
one or more UVC light sources with reflectors located on the robot base and controllable by the robot base to disinfect the site while the robot base navigates the site;
a portable self-contained disinfection unit configured to dock to the robot base and charge from the robot base, the portable self-contained disinfection unit comprising a portable UVC light source, battery and control circuitry, whereby the portable self-contained disinfection unit is configured to be removed from the robot base once charged and used by a user to execute an additional disinfection task; and
a processor configured to navigate the robot and while controlling the one or more UVC light sources to deliver a required UVC power for disinfection for disinfecting one or more surfaces within the site.

22. The robot in accordance with claim 21, wherein the portable self-contained disinfection unit further comprises a visual sensing system, an inertial measurement unit and a wireless radio communication system.

23. The robot in accordance with claim 21, wherein the portable self-contained disinfection unit further comprises a guidance facility to guide a user with respect to the additional disinfection task.

24. The robot in accordance with claim 21, wherein the guidance facility provides at least one of audio and visual guidance.
